(19) United States
(12) Patent Application Publication
(10) Pub. No.: US 2025/0082283 A1
(43) Pub. Date: Mar. 13, 2025

(54) Assistive Positioning Device For A Limb Of A Patient

(71) Applicant: United States Government as Represented by the Department of Veterans Affairs, Washington, DC (US)

(72) Inventors: Elizabeth A. Murdaugh, Charleston, SC (US); Joshua C. Dewitt, Charleston, SC (US)

(21) Appl. No.: 18/885,039

(22) Filed: Sep. 13, 2024

Related U.S. Application Data
(60) Provisional application No. 63/582,341, filed on Sep. 13, 2023.

(51) Int. Cl. A61B 6/04

(52) U.S. Cl. CPC A61B 6/0407

(57) ABSTRACT

An assistive positioning device for supporting an arm of a patient includes a assembly secured to a support surface, and includes a base riser assembly rotatably mounted to the base. An upper arm assembly has a first end mounted to the base riser assembly and a second end. An elbow assembly rotatably serves a lower arm assembly to the upper arm assembly. A handle portion is served to a distant end of the low arm assembly. An entirety of the assistive positioning device can be free of magnetic metal.

### CROSS-REFERENCE TO RELATED APPLICATION

The application claims priority to and the benefit of the filing date of U.S. Provisional Patent Application No. 63/582,341, filed Sep. 13, 2023, the entirety of which is hereby incorporated by reference herein.

### FIELD

The application is generally related to medical devices and procedures and, more particularly, to positioning devices and methods of use thereof that facilitate the positioning of a limb of a patient when imaging the limb.

### BACKGROUND

Positioning devices are well known for assisting medical personnel in positioning a patient's limb, such as an arm or leg, when taking x-rays. Preferably, a positioning device for a patient's limb will offer varying degrees of adjustability to accommodate patients of different sizes and physical capabilities. Known positioning devices are often fairly complex and include metal components. Although these devices are usually sufficient for use when taking images such as x-rays, the metal components make them incompatible with practices such as magnetic resonance imaging (MRI) due to the metal components. As such, it is not uncommon for a patient to have to maintain the position of their limb unassisted. Although this is acceptable for certain positions with certain patients, it can be severely limiting for some patients who lack the necessary strength to maintain the position of the limb during an imaging process.

From the foregoing, one recognizes the need for devices and methods of use thereof for the positioning of patients' limbs during various medical procedures.

### SUMMARY

Embodiments of the disclosed assistive positioning device for supporting an arm of a patient may include a base configured to releasably secure the positioning device to a support surface, such as an imaging table, and a base riser assembly including a first portion and a second portion. The base riser assembly allows manipulation of the positioning device as the first portion and the second portion are selectively rotatable with respect to each other, and the first portion of the base riser assembly is mounted to the base. The device also includes an upper arm assembly having a first end and a second end, the first end being mounted to the second portion of the base riser assembly, an elbow assembly comprising a first portion and a second portion, the first portion and the second portion of the elbow assembly are selectively rotatable with respect to each other. A lower arm assembly has a first end and a second end, the first end being mounted to the second portion of the elbow assembly, and the second end being connected to a handle portion. An entirety of the assistive positioning device can be free of magnetic metal.

In some embodiments, disclosed is an assistive positioning device for supporting an arm of a patient during an imaging process. The device may include a base configured to releasably secure the positioning device to a support surface, such as an imaging table. A base riser assembly includes a first portion and a second portion, the first portion and the second portion are selectively rotatable with respect to each other and the first portion of the base riser assembly is mounted to the base. The device also includes an upper arm assembly with an outer rod including a first end and a second end, the first end of the outer rod being mounted to the second portion of the base riser assembly, and an inner rod including a first end and a second end, wherein the inner rod is slidably received within the outer rod. A cam lock is configured to non-slidably fix the inner rod with respect to the outer rod. An elbow assembly includes a first portion and a second portion. The first portion is secured to the second end of the inner rod of the upper arm assembly, and the first portion and the second portion of the elbow assembly are selectively rotatable with respect to each other. A lower arm assembly has a first end and a second end, the first end is mounted to the second portion of the elbow assembly, and a handle portion is secured to a second end of the lower arm assembly.

Preferably, embodiments disclosed herein allow a large degree of adjustability to accommodate properly positioning the patient's limb during an imaging process. For example, preferred embodiments allow a patient's arm to be positioned anywhere from alongside their torso to vertically above their head such as when being passed through the MRI device. Additionally, embodiments disclosed herein can be constructed from non-metallic and/or non-ferrous materials so that they may pass through an MRI device during the process. As such, the disclosed devices, systems, and methods may lead to greater comfort for the patient as well as creation of better quality images during the MRI process.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A and 1B are top and side plan views of an assistive positioning device for positioning a limb of a patient in accordance with an embodiment as disclosed herein;

FIG. 1B is a side plan view of the assistive positioning device shown in FIG. 1;

FIG. 2 is an exploded view of the assistive positioning device shown in FIG. 1;

FIGS. 3A and 3B are perspective exploded views of the base assembly of the assistive positioning device shown in FIG. 1;

FIG. 4 is a cross-sectional view of the base assembly shown in FIGS. 3A and 3B;

FIG. 5 is an exploded view of the upper arm assembly of the assistive positioning device shown in FIG. 1;

FIG. 6 is an exploded view of the upper arm assembly shown in FIG. 5;

FIG. 7 is an exploded view of a cam lock of the assistive positioning device shown in FIG. 1;

FIG. 8 is a cross-sectional view of the cam lock shown in FIG. 7;

FIGS. 9A and 9B are exploded views of the elbow assembly of the assistive positioning device shown in FIG. 1;

FIG. 10 is an exploded view of the lower arm assembly of the assistive positioning device shown in FIG. 1; and

FIG. 11 is a perspective view of the assistive positioning device shown in FIG. 1 attached to a patient support surface of an MRI device.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein, usage the singular forms “a,” “an,” and “the” include disclosure of both singular and plural referents unless the context clearly dictates otherwise. For example, use of the term “a loop” can represent disclosure of embodiments in which only a single loop is provided, as well as embodiments in which a plurality of such loops is provided.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

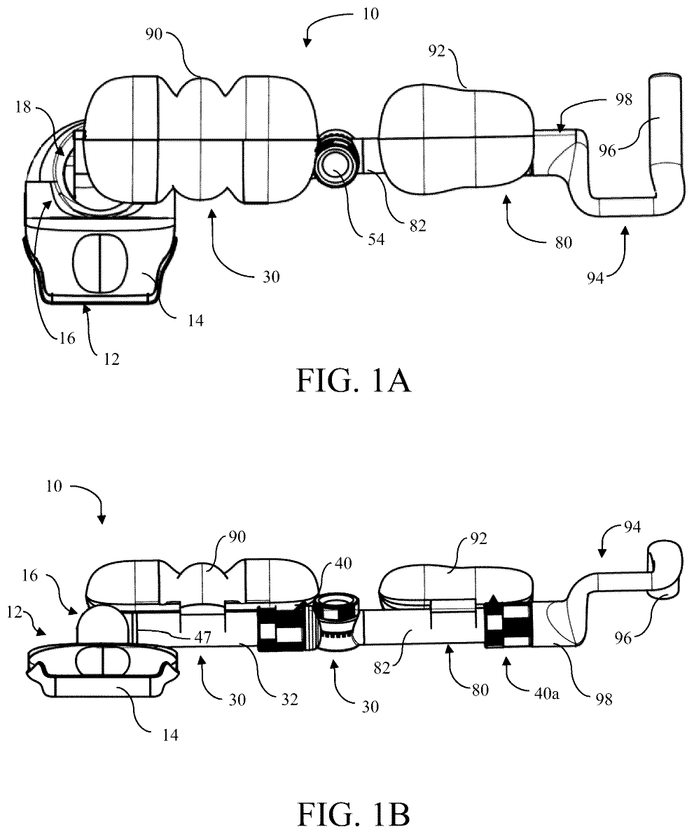

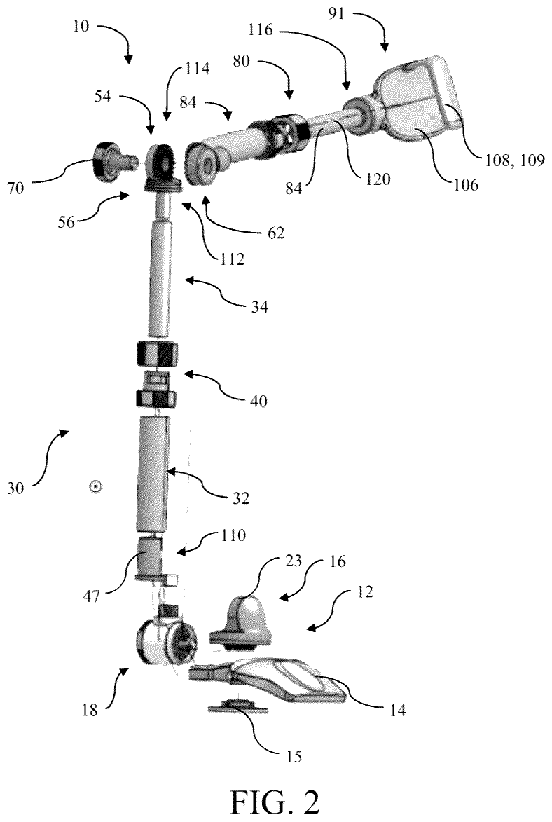

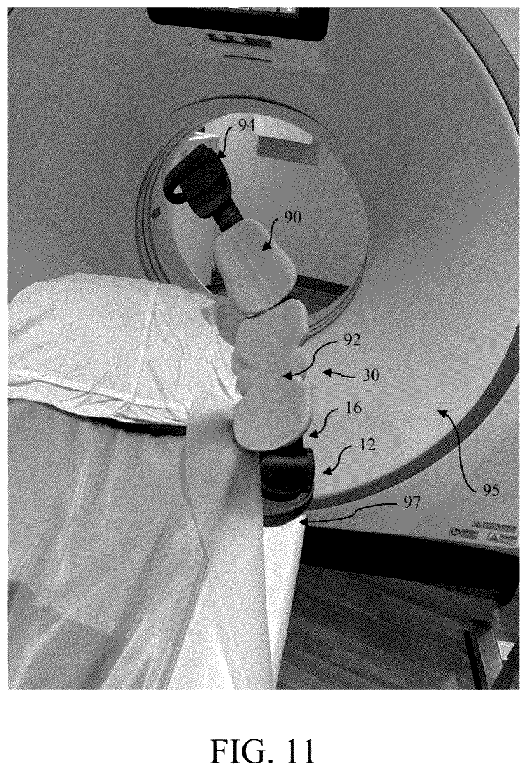

Referring now to the figures, a modular assisting positioning device 10 in accordance with an embodiment of the present disclosure is shown in FIGS. 1A, 1B, and 2. The modular assisting positioning device 10 may include a base assembly 12, a base swivel assembly 16 attached thereto, an upper arm assembly 30, and a lower arm assembly 80 that is connected to the upper arm assembly 30 by an adjustable elbow assembly 54. In some aspects, all components of the positioning device 10 are constructed of non-metallic and/or non-ferrous materials. Accordingly, the positioning device 10 can be essentially free of, or free of, magnetic metal (e.g., free of ferrous metal). Optionally, the positioning device 10 can be essentially free of, or free of metal. Accordingly, the positioning device 10 can consist of non-magnetic metal elements (optionally, consisting of non-metal components). As such, the positioning device 10 may be used in devices such as magnetic resonance imaging (MRI) systems 95 (FIG. 11), whereas known positioning devices that typically include components constructed from ferrous materials (e.g., fasteners, levers, and the like) may not.

The upper arm assembly 30 can have a first end 110 and a second end 112. The lower arm assembly 80 can have a first end 114 and a second end 116.

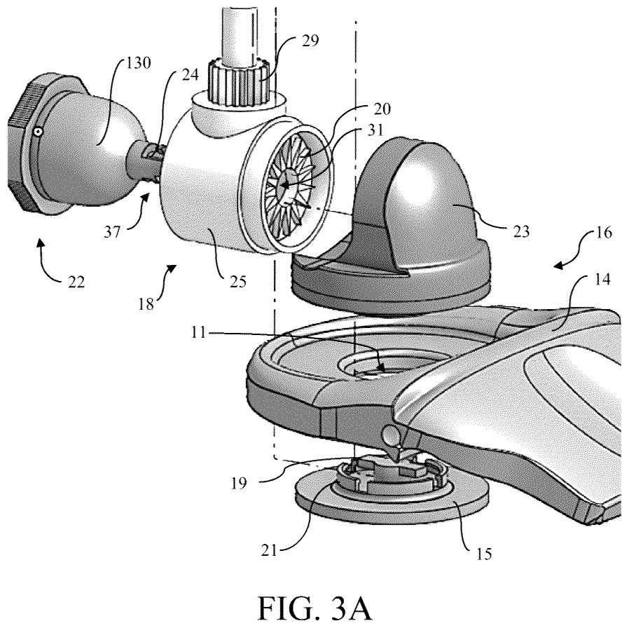

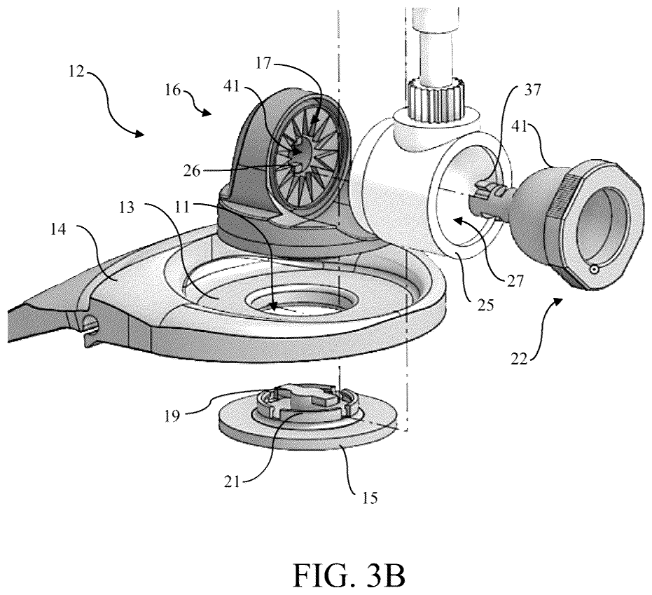

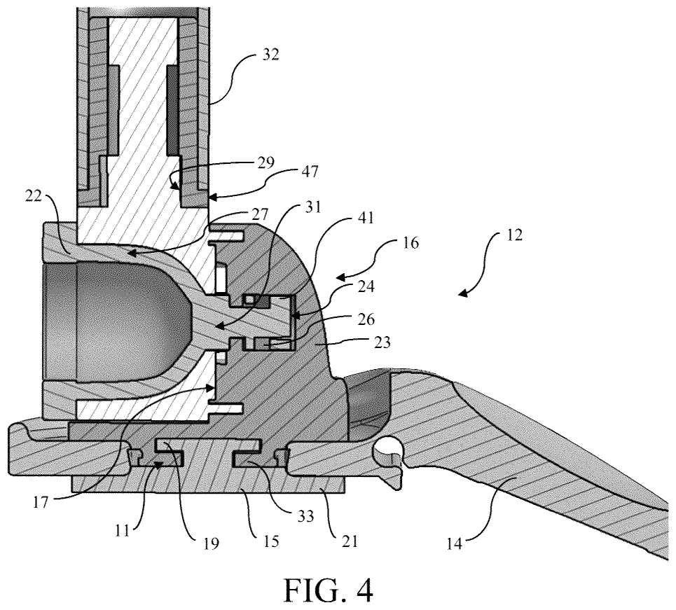

As best seen in FIGS. 3A, 3B and 4, the base assembly 12 includes a base adapter 14 that is configured to be removably secured to the side of a patient support surface, such as a bed 97 (FIG. 11), on which a patient rests when being placed in an MRI system 95. The base adaptor 14 includes a recessed portion 13 defining a bore 11 therein, the bore 11 being configured to rotationally receive the base swivel assembly 16 therein. The base swivel assembly 16 includes a base plate 15 having a central portion 21 (e.g., a cylindrical or substantially cylindrical central portion) and a pair of lock tabs 19. The base point 15 is received adjacent the bottom surface of the base adaptor 14 such that the central portion 21 of the base point 15 extends upwardly through the bore 11 of the base adaptor 14. The base swivel assembly 16 also includes a body portion 23 that includes a pair of lock slots 33 in which the lock tabs 19 of the base plate 15 are rotationally received, as best seen in FIG. 4. With the lock tabs 19 of the base plate 15 received in lock slots 33 of the body portion 23, the base swivel assembly 16 is preferably rotatable about 180 degrees with respect to the base adaptor 14 at any desired increment.

Still with reference to FIGS. 3A, 3B, and 4, the body portion 23 of the base swivel assembly 16 includes a toothed face 17 and is configured to engage a correspondingly toothed face 20 of the base riser 25 to form a base riser assembly 18. As such, the base riser 25 includes a cylindrical or substantially cylindrical body portion defining the toothed face 20 on one end and a bore 27 on the opposite end. As shown, the base riser 25 is adjustably secured to the body portion 23 of the base swivel assembly 16 by a key 22 that includes a body portion 130 and a projection 37 (e.g., a cylindrical projection) extending outwardly therefrom. The projection 37 defines the locking tracks 24 in its outer surface and is configured to pass through an aperture 31 that extends from the bore 27 through the toothed face 20. The projection 37 is configured to be both slidably and rotatably received within a blind bore 41 that extends inwardly from the toothed face 17 of the body portion 23 of the base swivel assembly 16. A pair of lock tabs 26 extend inwardly from the walls of the blind bore 41 of the body portion 23 of the base swivel assembly 16 and are configured to be slidably received in the locking tracks 24 on the outer surface of the projection 37 of the key 22. With the projection 37 of the key 22 removed from the blind bore 41, the base riser 25 is rotatable with respect to the body portion 23 of the base swivel assembly 16 over a range of approximately 180 degrees at increments of 24 degrees. Note, however, other desired increments may be selected based on the size and number of teeth on the toothed faces 20 and 17. When the desired position is reached, the key 22 is used to engage the toothed faces 20 and 17 of the base riser 25 and the body portion 23 of the base swivel assembly 16, respectively, in the desired position.

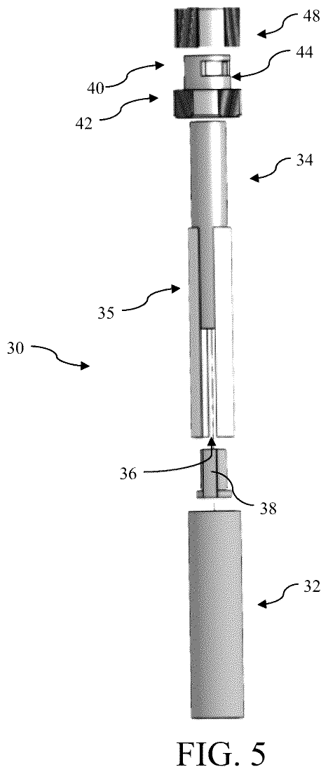

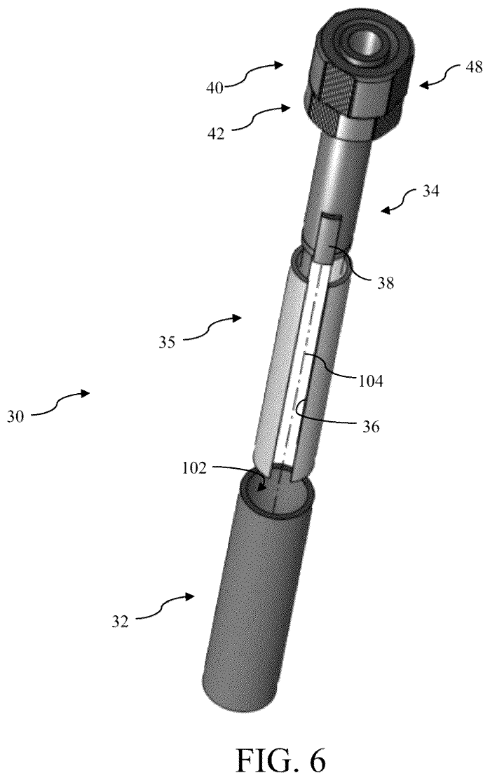

Referring now to FIGS. 5 and 6, the upper arm assembly 30 of the positioning device 10 preferably includes an outer rod 32, an inner-rod 34 that is slidably disposed therein, a pair of guide lock tabs 38 that interact with a pair of guide slots 36, and an upper arm pad 90. The guide slots 36 are preferably formed by a pair of guide lock tracks 35 that are non-rotatably secured to the inner surface 102 of the outer rod 32. As shown, the outer rod 32 can be cylindrical or substantially cylindrical and can include a first end that is received on a toothed projection 29 that extends outwardly from the body portion 25 of the base riser assembly 18, as shown in FIG. 4. In some aspects, pair of guide lock tracks 35 can extend along the inner surface 102 of the outer rod 32 parallel to a longitudinal center axis 104 of the outer rod. A rotation insert 47 is disposed between the first end of the outer rod 32 and the toothed projection 29. The rotation insert 47 allows the upper arm assembly 30 to be rotated over a range of 360 degrees at 18 degree increments with respect to the base riser assembly 18. A cam lock assembly 40 is fixed to the opposite second end of the outer rod 32 of the upper arm assembly 30, as discussed in greater detail below.

As shown, the pair of guide lock tracks 35 is disposed within the outer rod 32 of the upper arm assembly 30 so that they define a pair of axially elongated slots 36 therebetween. The guide lock tracks 35 may be secured to the inner surface of the outer rod 32 by adhesives or fasteners, or any appropriate known method. Each elongated axial slot 36 is configured to slidably receive a guide lock tab 38 therein. The pair of guide lock tabs 38 is affixed to one end of the inner rod 34 of the upper arm assembly 30. As shown, the guide lock tabs 38 extend outwardly from a plug that is received in an open end of the inner rod 34. The upper arm pad 90 is removably secured to the outer rod 32 of the upper arm assembly 30. Preferably, the upper arm pad 90 is rotatable about, and axially slidable along, the outer rod 32 so that a patient's upper arm may be supported thereon regardless of the position of the upper arm assembly 30.

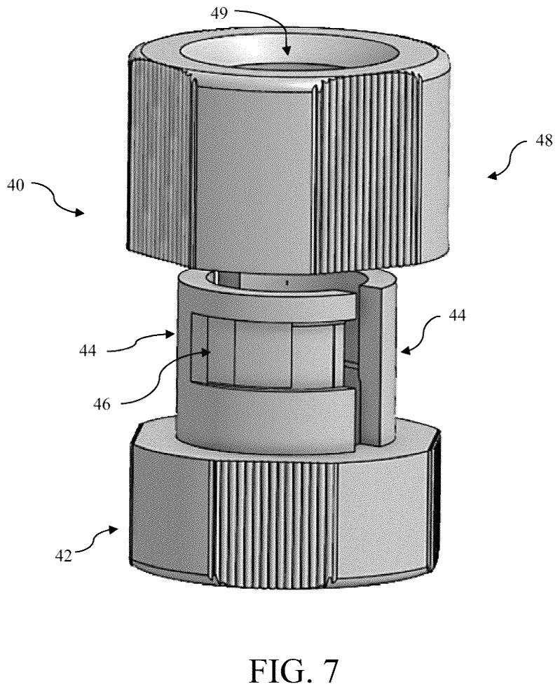

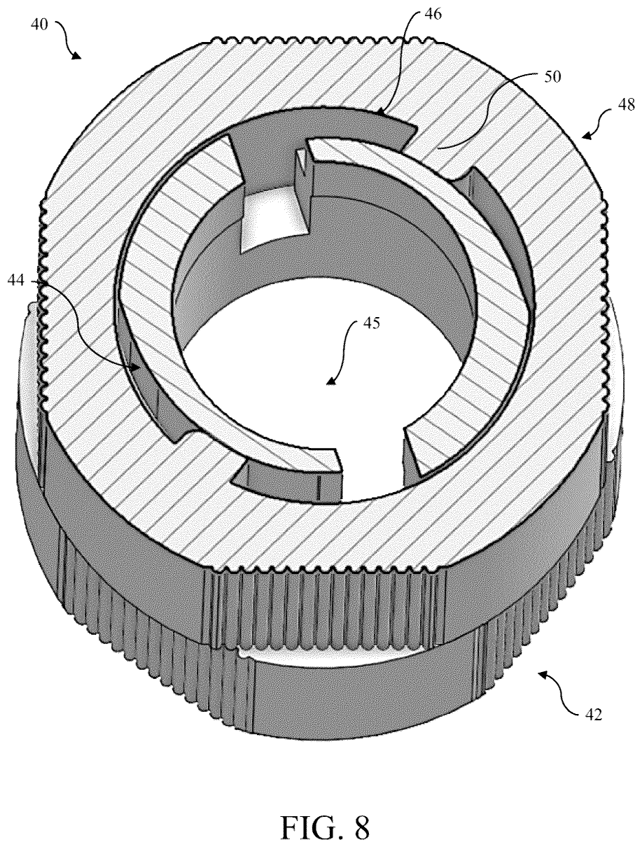

Referring additionally to FIGS. 7 and 8, a cam lock assembly 40 in accordance with the present disclosure includes a male portion 42 including a pair of lock walls 44 that define a central projection, each lock wall 44 including a lock slot 46 formed therein. The male portion 42 of the cam lock assembly 40 is fixed to the second end of the outer rod 32 of the upper arm assembly 30. The male portion 42 of the cam lock assembly 40 defines a central bore 45 that has a diameter that is slightly larger than the outer diameter of the inner rod 34 of the upper arm assembly 30. As such, the inner rod 34 of the upper arm assembly 30 is slidably receivable through the bore 45 of the male portion 42. The cam lock assembly 40 also includes a female portion 48 that defines a central bore 49 that rotatably receives the semi-cylindrical lock walls 44 of the male portion 42 therein. As shown, the bore 49 of the female portion 48 includes a pair of inwardly depending lock tabs 50 that are slidably received in the lock slots 46 defined in the outer surfaces of the lock walls 44 of the male portion 42.

As best seen in FIG. 8, each lock slot 46 of the male portion 42 gets progressively deeper as a corresponding lock tab 50 moves in a counter-clockwise (CCW) direction with respect to the lock walls 44. As such, rotation of the female portion 48 in the CCW direction releases inward pressure on the lock walls 44 by the lock tabs 50. As such, the lock walls 44 deflect outwardly away from contact with the inner rod 34 so the inner rod 34 may be slidably adjusted. Conversely, rotation of the female portion 48 in the CW direction with regard to the male portion 42 of the cam lock assembly 40 causes the lock walls 44 to be urged inwardly by the lock tabs 50. Eventually, the lock walls 44 engage the outer surface of the inner rod 34 of the upper arm assembly 30, thereby locking the inner rod 34 in place. The desired overall length of the upper arm assembly 30 is selected by loosening and tightening the cam lock assembly 40 as desired as the inner rod 34 of the upper arm assembly 30 is axially slid with respect to the outer rod 32. As previously discussed, the guide lock tabs 38 that are affixed to the inner rod 34 engage the slots 36 defined by the guide lock tracks 35, thereby preventing rotation of the inner rod 34 with respect to the outer rod 32.

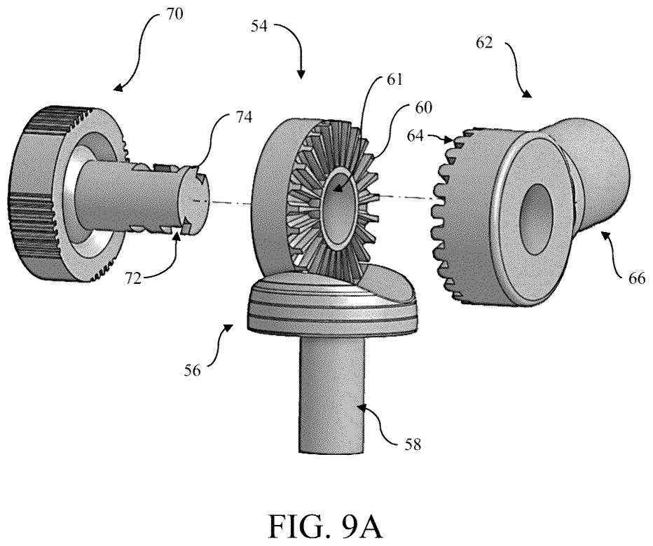

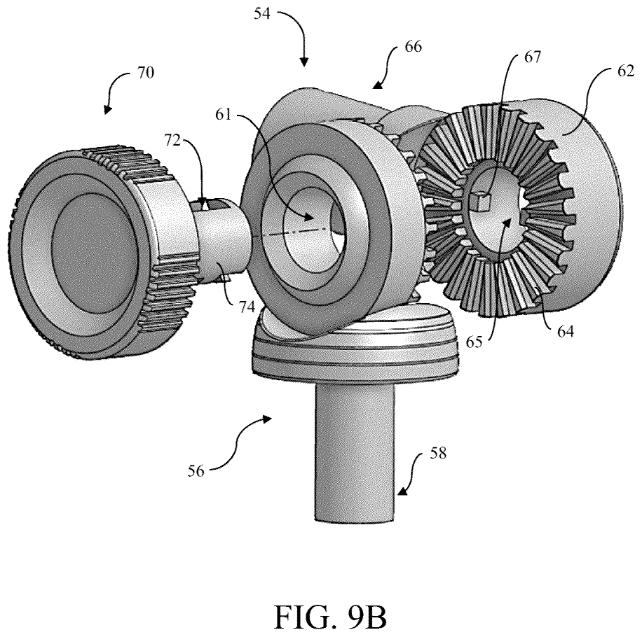

Referring now to FIGS. 2, 9A, and 9B, the lower arm assembly 80 is adjustably secured to the upper arm assembly 30 by an elbow assembly 54. As shown, the elbow assembly 54 includes an upper arm adapter 56 and a lower arm adapter 62 that are secured to each other by a locking key 70. The elbow assembly 54 is secured to the upper arm assembly 30 by the upper arm adapter 56. The upper arm adapter 56 includes a body portion having a mounting post 58 (e.g., a cylindrical or substantially cylindrical mounting post) that is non-rotatably received in the second end of the inner rod 34 of the upper arm assembly 30. The body portion can also define a central bore 61 (e.g., a cylindrical or substantially cylindrical central bore) that is surrounded by a toothed face 60 at one end of the bore 61. The toothed face 60 of the upper arm adapter 56 is configured to selectively engage a correspondingly toothed face 64 of the lower arm adapter 62. The lower arm adapter 62 includes a body portion having a mounting post 66 that is configured to be non-rotatably received in a first end of an outer rod 82 of the lower arm assembly 80, as discussed below. As noted, the lower arm adapter 62 is adjustably secured to the upper arm adapter 56 by a locking key 70 that includes a body portion with a projection 74 (e.g., a cylindrical or substantially cylindrical projection) that defines locking tracks 72 in its outer surface. The projection 74 is configured to pass through the central bore 61 of the upper arm adapter 56 at the center of its toothed face 60. The projection 74 is also configured to be both slidably and rotatably received within a central bore 65 that is defined at the center of the toothed face 64 of the lower arm adapter 62. A pair of lock tabs 67 extend inwardly from the walls of the central bore 65 of the lower arm adapter 62 and are configured to be slidably received in the locking tracks 72 of the projection 74 of the locking key 70. With the projection 74 of the locking key 70 removed from the bore 65 of the lower arm adapter 62, the lower arm assembly 80 is rotatable with respect to the upper arm assembly 30 over a range of approximately 90 degrees in 15 degree increments. Note, however, that the size of the increments may be changed based on the size and spacing of the teeth on the toothed faces 60 and 64 of the upper arm adapter 56 and the lower arm adapter 62, respectively. When the desired position of the lower arm assembly 80 with respect to the upper arm assembly 30 is reached, the lock key 70 is used to engage the toothed faces 60 and 64 in the desired positions.

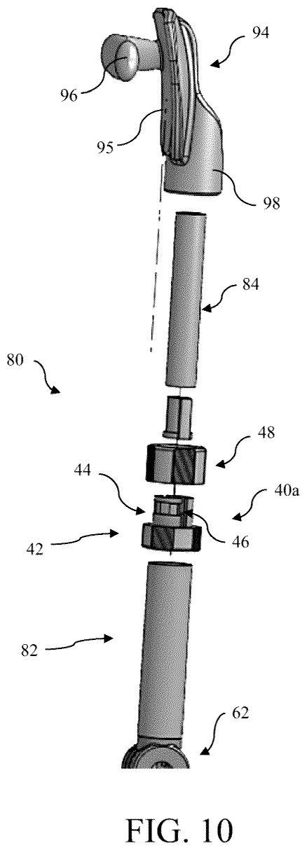

Referring now to FIG. 10, the lower arm assembly 80 of the positioning device 10 preferably includes an outer rod 82, an inner rod 84 that is slidably and rotatably disposed therein, a lower arm pad 92, and a hand support 94 affixed to the distal second end of the inner rod 84. As shown, the outer rod 82 can be cylindrical or substantially cylindrical and can include a first end that is non-rotatably received on the mounting post 66 that extends outwardly from the body portion of the lower arm adaptor 62, as shown in FIG. 9A. A cam lock assembly 40, as shown in FIGS. 7 and 8, is fixed to the opposite second end of the outer rod 82 of the lower arm assembly 80. Unlike the upper arm assembly 30, no guide lock tracks 35 (FIG. 5) are mounted inside the outer arm 82 of the lower arm assembly 80. As such, the inner rod 84 of the lower arm assembly 80 is able to be rotated with respect to the outer rod 82. The lower arm pad 92 can be removably secured to the outer rod 82 of the lower arm assembly 80. Preferably, the lower arm pad 92 is rotatable about, and axially slidable along, the outer rod 82 so that a patient's lower arm may be supported thereon regardless of the position of the lower arm assembly 80.

Referring again to FIGS. 7 and 8, a cam lock assembly 40a mounted to the second end of the outer rod 82 of the lower arm assembly 80 is practically identical to the previously discussed cam lock assembly 40. The cam lock assembly 40a includes a male portion 42 including a pair of lock walls 44 that define a central projection, each lock wall 44 including a lock slot 46 formed therein. The male portion 42 of the cam lock assembly 40 is fixed to the second end of the outer rod 82 of the lower arm assembly 80. The male portion 42 of the cam lock assembly 40 defines a central bore 45 that has a diameter that is slightly larger than the outer diameter of the inner rod 84 of the lower arm assembly 80. As such, the inner rod 84 of the lower arm assembly 80 is slidably receivable through the bore 45 of the male portion 42. The cam lock assembly 40 also includes a female portion 48 that defines a central bore 49 that rotatably receives the semi-cylindrical lock walls 44 of the male portion 42 therein. As shown, the bore 49 of the female portion 48 includes a pair of inwardly depending lock tabs 50 that are slidably received in the lock slots 46 defined in the outer surfaces of the lock walls 44 of the male portion 42.

As previously discussed, each lock slot 46 of the male portion 42 gets progressively deeper as a corresponding lock tab 50 moves in a counter-clockwise (CCW) direction with respect to the lock walls 44. As such, rotation of the female portion 48 in the CCW direction releases inward pressure on the lock walls 44 by the lock tabs 50. As such, the lock walls 44 deflect outwardly away from contact with the inner rod 84 so the inner rod 84 may be both slidably and rotatably adjusted. Conversely, rotation of the female portion 48 in the CW direction with regard to the male portion 42 of the cam lock assembly 40 causes the lock walls 44 to be urged inwardly by the lock tabs 50. As such, the lock walls 44 eventually engage the outer surface of the inner rod 84 of the lower arm assembly 80, thereby locking the inner rod 84 in place.

As best seen in FIG. 10, the hand support 94 includes a body portion 98 that is affixed to the second end of the inner rod 84 of the lower arm assembly 80. Although the hand support 94 is non-rotatably fixed to the inner rod 84, the inner rod 84 is selectively rotatable with respect to the outer rod 82 of the lower arm assembly 80. As such, the hand support 94 is both axially and rotatably adjustable as desired. A handle portion 96 is also provided as an easy grip for patients to grasp as necessary when positioning the arm. The handle portion 96 can comprise a base portion 106 secured to the second end 116 of the lower arm assembly 80 and a handle 108 for grasping that is formed by an elongated bar 109 that is transverse to a longitudinal center axis 120 of the lower arm assembly.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: An assistive positioning device for supporting an arm of a patient during an imaging process, comprising:

a base configured to releasably secure the positioning device to a support surface;

a base riser assembly comprising a first portion and a second portion, wherein the first portion and the second portion are selectively rotatable with respect to each other, and wherein the first portion of the base riser assembly is mounted to the base;

an upper arm assembly having a first end and a second end, the first end being mounted to the second portion of the base riser assembly;

an elbow assembly comprising a first portion and a second portion, wherein the first portion of the elbow assembly is secured to the second end of the upper arm assembly, and the first portion and the second portion of the elbow assembly are selectively rotatable with respect to each other;

a lower arm assembly having a first end and a second end, the first end being mounted to the second portion of the elbow assembly; and

a handle portion secured to a second end of the lower arm assembly,

wherein an entirety of the assistive positioning device is free of magnetic metal.
Aspect 2: The assistive positioning device of aspect 1, wherein the upper arm assembly further comprises:

an outer rod including a first end and a second end, the first end of the outer rod being mounted to the second portion of the base riser assembly; and

an inner rod including a first end and a second end, wherein the inner rod is slidably received within the outer rod and the second end of the inner rod is secured to the first portion of the elbow assembly.
Aspect 3: The assistive positioning device of aspect 2, wherein the inner rod is non-rotatably secured to the outer rod.
Aspect 4: The assistive positioning device of aspect 3, wherein the upper arm assembly further comprises:

at least one pair of guide tracks defining an elongated groove, the at least one pair of guide tracks extending along an inner surface of the outer rod parallel to a longitudinal center axis of the outer rod; and

at least one projection extending outwardly from an outer surface of the inner rod, the at least one projection being slidably received in the elongated groove so that the inner rod is non-rotatable with respect to the outer rod.
Aspect 5: The assistive positioning device of aspect 3, wherein the upper arm assembly further comprises a cam lock that is configured to non-slidably fix the inner rod with respect to the outer rod.
Aspect 6: The assistive positioning device of aspect 5, wherein the cam lock further comprises a first portion secured to the second end of the outer rod of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.
Aspect 7: The assistive positioning device of aspect 6, wherein the first portion of the cam lock includes a cam portion that is movable between a first outer position that is spaced from an outer surface of the inner rod when the second portion of the cam lock is in the first position, and a second inner position abutting the outer surface of the inner rod when the second portion of the cam lock is in the second position.
Aspect 8: The assistive positioning device of aspect 6, wherein the lower arm assembly further comprises:

an outer rod including a first end and a second end, the first end of the outer rod being mounted to the second portion of the elbow assembly; and

an inner rod including a first end and a second end, wherein the inner rod is slidably received within the outer rod of the lower arm assembly and the second end of the inner rod is secured to the handle portion.
Aspect 9: The assistive positioning device of aspect 8, wherein the lower arm assembly further comprises a cam lock that is configured to non-slidably fix the inner rod with respect to the outer rod.
Aspect 10: The assistive positioning device of aspect 9, wherein the cam lock further comprises a first portion secured to the second end of the outer rod of the lower arm assembly of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.
Aspect 11: The assistive positioning device of aspect 1, wherein the handle portion is rotatable with respect to the lower arm assembly.
Aspect 12: The assistive positioning device of aspect 11, wherein the handle portion comprises a base portion secured to the second end of the lower arm assembly, and a handle for grasping that is formed by an elongated bar that is transverse to a longitudinal center axis of the lower arm assembly.
Aspect 13: An assistive positioning device for supporting an arm of a patient during an imaging process, comprising:

a base configured to releasably secure the positioning device to a support surface;

a base riser assembly comprising a first portion and a second portion, wherein the first portion and the second portion are selectively rotatable with respect to each other, and the first portion of the base riser assembly is mounted to the base;

an upper arm assembly comprising:
an outer rod including a first end and a second end, the first end of the outer rod being mounted to the second portion of the base riser assembly;
an inner rod including a first end and a second end, wherein the inner rod is slidably received within the outer rod; and
a cam lock that is configured to non-slidably fix the inner rod with respect to the outer rod;

an elbow assembly comprising a first portion and a second portion, wherein the first portion of the elbow assembly is secured to the second end of the inner rod of the upper arm assembly, and the first portion and the second portion of the elbow assembly are selectively rotatable with respect to each other;

a lower arm assembly having a first end and a second end, the first end being mounted to the second portion of the elbow assembly; and

a handle portion secured to a second end of the lower arm assembly.
Aspect 14: The assistive positioning device of aspect 13, wherein an entirety of the assistive positioning device is free of magnetic metal.
Aspect 15: The assistive positioning device of aspect 14, wherein the inner rod of the upper arm assembly is non-rotatably secured to the outer rod.
Aspect 16: The assistive positioning device of aspect 14, wherein the cam lock further comprises a first portion secured to the second end of the outer rod of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.
Aspect 17: The assistive positioning device of aspect 16, wherein the first portion of the cam lock includes a cam portion that is movable between a first outer position that is spaced from an outer surface of the inner rod when the second portion of the cam lock is in the first position, and a second inner position abutting the outer surface of the inner rod when the second portion of the cam lock is in the second position.
Aspect 18: The assistive positioning device of aspect 13, wherein the handle portion is rotatable with respect to the lower arm assembly.
Aspect 19: The assistive positioning device of aspect 18, wherein the handle position comprises a base portion secured to the second end of the lower arm assembly, and a handle for grasping that is formed by an elongated bar that is transverse to a longitudinal center axis of the lower arm assembly.
Aspect 20: The assistive positioning device of aspect 13, wherein the upper arm assembly further comprises:

at least one pair of guide tracks defining an elongated groove, the at least one pair of guide tracks extending along an inner surface of the outer rod parallel to a longitudinal center axis of the outer rod; and

at least one projection extending outwardly from an outer surface of the inner rod, the at least one projection being slidably received in the elongated groove so that the inner rod is non-rotatable with respect to the outer rod.

Although the foregoing invention has been described in some detail by way of illustration and example for purposes of clarity of understanding, certain changes and modifications may be practiced within the scope of the appended claims.

What is claimed is:

1. An assistive positioning device for supporting an arm of a patient during an imaging process, comprising:
a base configured to releasably secure the positioning device to a support surface;
a base riser assembly comprising a first portion and a second portion, wherein the first portion and the second portion are selectively rotatable with respect to each other, and wherein the first portion of the base riser assembly is mounted to the base;
an upper arm assembly having a first end and a second end, the first end being mounted to the second portion of the base riser assembly;
an elbow assembly comprising a first portion and a second portion, wherein the first portion of the elbow assembly is secured to the second end of the upper arm assembly, and the first portion and the second portion of the elbow assembly are selectively rotatable with respect to each other;
a lower arm assembly having a first end and a second end, the first end being mounted to the second portion of the elbow assembly; and
a handle portion secured to a second end of the lower arm assembly,
wherein an entirety of the assistive positioning device is free of magnetic metal.

2. The assistive positioning device of claim 1, wherein the upper arm assembly further comprises:
an outer rod including a first end and a second end, the first end of the outer rod being mounted to the second portion of the base riser assembly; and
an inner rod including a first end and a second end, wherein the inner rod is slidably received within the outer rod and the second end of the inner rod is secured to the first portion of the elbow assembly.

3. The assistive positioning device of claim 2, wherein the inner rod is non-rotatably secured to the outer rod.

4. The assistive positioning device of claim 3, wherein the upper arm assembly further comprises:
at least one pair of guide tracks defining an elongated groove, the at least one pair of guide tracks extending along an inner surface of the outer rod parallel to a longitudinal center axis of the outer rod; and
at least one projection extending outwardly from an outer surface of the inner rod, the at least one projection being slidably received in the elongated groove so that the inner rod is non-rotatable with respect to the outer rod.

5. The assistive positioning device of claim 3, wherein the upper arm assembly further comprises a cam lock that is configured to non-slidably fix the inner rod with respect to the outer rod.

6. The assistive positioning device of claim 5, wherein the cam lock further comprises a first portion secured to the second end of the outer rod of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.

7. The assistive positioning device of claim 6, wherein the first portion of the cam lock includes a cam portion that is movable between a first outer position that is spaced from an outer surface of the inner rod when the second portion of the cam lock is in the first position, and a second inner position abutting the outer surface of the inner rod when the second portion of the cam lock is in the second position.

8. The assistive positioning device of claim 6, wherein the lower arm assembly further comprises:
an outer rod including a first end and a second end, the first end of the outer rod being mounted to the second portion of the elbow assembly; and
an inner rod including a first end and a second end, wherein the inner rod is slidably received within the outer rod of the lower arm assembly and the second end of the inner rod is secured to the handle portion.

9. The assistive positioning device of claim 8, wherein the lower arm assembly further comprises a cam lock that is configured to non-slidably fix the inner rod with respect to the outer rod.

10. The assistive positioning device of claim 9, wherein the cam lock further comprises a first portion secured to the second end of the outer rod of the lower arm assembly of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.

11. The assistive positioning device of claim 1, wherein the handle portion is rotatable with respect to the lower arm assembly.

12. The assistive positioning device of claim 11, wherein the handle portion comprises a base portion secured to the second end of the lower arm assembly, and a handle for grasping that is formed by an elongated bar that is transverse to a longitudinal center axis of the lower arm assembly.

13. An assistive positioning device for supporting an arm of a patient during an imaging process, comprising:
a base configured to releasably secure the positioning device to a support surface;
a base riser assembly comprising a first portion and a second portion, wherein the first portion and the second portion are selectively rotatable with respect to each other, and the first portion of the base riser assembly is mounted to the base;
an upper arm assembly comprising:
an outer rod including a first end and a second end, the first end of the outer rod being mounted to the second portion of the base riser assembly;
an inner rod including a first end and a second end, wherein the inner rod is slidably received within the outer rod; and
a cam lock that is configured to non-slidably fix the inner rod with respect to the outer rod;
an elbow assembly comprising a first portion and a second portion, wherein the first portion of the elbow assembly is secured to the second end of the inner rod of the upper arm assembly, and the first portion and the second portion of the elbow assembly are selectively rotatable with respect to each other;
a lower arm assembly having a first end and a second end, the first end being mounted to the second portion of the elbow assembly; and
a handle portion secured to a second end of the lower arm assembly.

14. The assistive positioning device of claim 13, wherein an entirety of the assistive positioning device is free of magnetic metal.

15. The assistive positioning device of claim 14, wherein the inner rod of the upper arm assembly is non-rotatably secured to the outer rod.

16. The assistive positioning device of claim 14, wherein the cam lock further comprises a first portion secured to the second end of the outer rod of the upper arm assembly and a second portion rotatably secured to the first portion of the cam lock, and wherein the second portion of the cam lock is rotatable between a first position in which the inner rod is slidable with respect to the outer rod, and a second position in which the inner rod is non-slidably fixed with respect to the outer rod.

17. The assistive positioning device of claim 16, wherein the first portion of the cam lock includes a cam portion that is movable between a first outer position that is spaced from an outer surface of the inner rod when the second portion of the cam lock is in the first position, and a second inner position abutting the outer surface of the inner rod when the second portion of the cam lock is in the second position.

18. The assistive positioning device of claim 13, wherein the handle portion is rotatable with respect to the lower arm assembly.

19. The assistive positioning device of claim 18, wherein the handle position comprises a base portion secured to the second end of the lower arm assembly, and a handle for grasping that is formed by an elongated bar that is transverse to a longitudinal center axis of the lower arm assembly.

20. The assistive positioning device of claim 13, wherein the upper arm assembly further comprises:
at least one pair of guide tracks defining an elongated groove, the at least one pair of guide tracks extending along an inner surface of the outer rod parallel to a longitudinal center axis of the outer rod; and
at least one projection extending outwardly from an outer surface of the inner rod, the at least one projection being slidably received in the elongated groove so that the inner rod is non-rotatable with respect to the outer rod.
